(19) United States
(12) Patent Application Publication
(10) Pub. No.: US 2020/0038305 A1
(43) Pub. Date: Feb. 6, 2020

(54) Sunscreen or Sunblock Composition

(71) Applicant: Divios LLC, Santa Barbara, CA (US)

(72) Inventors: Nathan Garrison, Santa Barbara, CA (US); David Garrison, Mt. Pleasant, SC (US); Davis Mersereau, Playa Del Rey, CA (US); Monica Vialpando, San Francisco, CA (US)

(21) Appl. No.: 16/530,738

(22) Filed: Aug. 2, 2019

Related U.S. Application Data
(60) Provisional application No. 62/713,766, filed on Aug. 2, 2018.

(51) Int. Cl. A61K 8/49; A61K 8/9789; A61Q 17/04; A61K 8/92; A61K 8/27; A61K 8/29

(52) U.S. Cl. CPC A61K 8/498; A61K 8/9789; A61K 8/29; A61K 8/922; A61K 8/27; A61Q 17/04

(57) ABSTRACT

The invention provides a sunscreen composition comprising cannabis extract. The invention also provides a method of protecting a substrate from the effect of ultraviolet light comprising administering an effective amount of a sunscreen composition comprising cannabis extract.

### CROSS-REFERENCE TO PRIOR APPLICATIONS

This application claims the benefit of U.S. Provisional Application Ser. No. 62/713,766, filed Aug. 2, 2018, the disclosure of which is incorporated by reference herein.

### BACKGROUND OF THE INVENTION

### Area of the Art

Embodiments of the present invention relate to improved sunscreen or sunblock compositions comprising cannabinoids.

The cannabis plant has many naturally occurring substances that are of interest in medicine. Isolated compounds from the cannabis plant include tetrahydrocannabinol (THC), cannabidiol (CBD), cannabichromene (CBC), cannabigerol (CBG), cannabidivarin (CBDV), among other compounds. Some isolated compounds from the cannabis plant are characterized as cannabinoids. A cannabinoid is also defined as a class of diverse chemical compounds that interact with the endocannabinoid system in cells. There are over one hundred and forty four (144) cannabinoids that have been identified in extracts from the cannabis plant. Many studies have confirmed the medicinal value of cannabinoids. Cannabinoids have been investigated for possible treatment of seizures, nausea, vomiting, lack of appetite, pain, arthritis, inflammation, and other conditions.

Cannabinoids are associated with a number of desirable properties including recognized medical benefits and intoxicant effects. The inventors of the present application have surprisingly shown that certain cannabinoids are surprisingly well suited to the absorption of UV wavelengths that are harmful to humans. In particular, the inventors have discovered that certain cannabinoids are surprisingly effective as a component in a sunscreen or sunblock composition.

### DESCRIPTION OF THE BACKGROUND ART

The present invention relates to the use of cannabis extract in sunscreen or sunblock compositions. In one embodiment, the invention relates to the use of cannabinoids such as tetrahydrocannabinol (THC) and cannabidiol (CBD) in sunscreen or sunblock compositions.

Solar ultraviolet (UV) radiation having a wavelength between 200 nm and 400 nm is commonly further classified into three regions: from 320 to 400 nm (UVA); from 290 to 320 nm (UVB); and from 200 to 290 nm (UVC). UVC radiation is largely absorbed by the ozone layer of the earth's atmosphere. However exposure of the skin to UVA and UVB radiation can result in skin reddening and skin burns. Moreover, prolonged exposure to UVA and UVB radiation can lead to the development of more serious skin disorders, such as light-induced dermatoses and erythemata (sunburn), as well as increasing the risk of skin cancers, including melanoma, as well as accelerating skin aging, for example, as a result of loss of skin elasticity and skin wrinkling Skin damage with UV exposure occurs through at least two mechanisms: (i) direct damage to cellular DNA, mainly from UVB (290-320 nm), and (ii) production of free radicals from UVA (320-400 nm), which can damage intracellular components including nucleic acids, biomembranes, and proteins.

Sunscreen or sunblock compositions capable of shielding the body from UVA and UVB radiation, and techniques of making such compositions are highly desirable. Nevertheless, although a plethora of sunscreen compositions has been developed, numerous shortcomings associated with known sunscreen compositions remain.

In order to evaluate the level of protection the skin receives when exposed to UVA and UVB radiation, sunscreen compositions are commonly assayed by measuring the sun protection factor (SPF) of the composition. Manufacturers attempt to provide consumers with sunscreen products having high SPF values. One way of achieving high SPF values is by incorporating significant concentrations of UVA and UVB sunscreen active ingredients into sunscreen products. These agents either absorb or reflect the UV rays before they can cause damage. However, above certain concentrations, safety, economic and regulatory limitations can become prohibitive. Furthermore, at high concentrations, sunscreen active ingredients may cause irritation, which is a particularly a concern when the sunscreen composition is applied to the facial area, where irritation of the delicate skin surrounding the eyes can readily occur. Furthermore, sweat can carry the composition into the eyes where severe irritation can occur. Sensory properties and aesthetics of sunscreen products can also be affected by the incorporation of high concentrations of sunscreen active ingredients. One further drawback to the approach of simply increasing the concentration of known sunscreen agents is the cost associated with the incorporation of high concentrations of sunscreen active ingredients, which may be expensive. Hence, there remains a problem in the art with providing sunscreen formulations exhibiting high SPF values and desirable sensory and aesthetic attributes, while having relatively low concentration of sunscreen active ingredients in the compositions.

Although a number of new substances have been investigated for use in a sunscreen or sunblock composition, many of those substances have been found to have toxic effects, so limits have been placed on the concentrations of those materials in a number of jurisdictions around the world. For example, in the case of para-aminobenzoic acid (PABA), the negative effects are so great that the substance has been entirely banned in the European Union.

It is desirable to develop sunscreen or sunblock compositions that comprise lower concentration of active ingredients such as titanium dioxide and zinc oxide but include natural ingredients, such as cannabinoids, that enhance the sunscreen or sunblock properties of the product.

The inventors of the present application have surprisingly shown that certain cannabinoids are surprisingly well suited to the absorption of UV wavelengths that are harmful to humans. In particular, the inventors have discovered that certain cannabinoids are surprisingly effective as a component in a sunscreen or sunblock composition. For example, even relatively low levels of certain cannabinoids can have profound effects on the efficacy of a sunscreen or sunblock composition.

Further objectives and advantages, as well as the structure and function of preferred embodiments will become apparent from a consideration of the description and examples.

### SUMMARY OF THE INVENTION

In accordance with the invention, provided is a sunscreen composition comprising a dermatologically acceptable carrier and a cannabis extract, wherein the composition protects a substrate from the effects of ultraviolet light. In one embodiment, the cannabis extract comprises a mixture of cannabinoids. In other embodiments, the mixture of cannabinoids comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

In one embodiment, the mixture of cannabinoids comprises any two components selected from tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran. In another embodiment, the mixture of cannabinoids comprises any two components in a ratio of about 1:20, about 1:10, or about 1:1.

In one embodiment, the mixture of cannabinoids comprises tetrahydrocannabinol and cannabidiol. In another embodiment, the mixture of cannabinoids comprises tetrahydrocannabinol and cannabidiol in a ratio of 3:1.

In other embodiments, the cannabis extract comprises between about 0.1 to about 20% weight/weight of the sunscreen composition. In still other embodiments, the sunscreen composition provides an SPF value of at least 5.

In one embodiment, the composition protects the substrate from the effect of ultraviolet light. In another embodiment, the composition protects the substrate from the effect of ultraviolet A light. In still another embodiment, the composition protects the substrate from the effect of ultraviolet B light.

In accordance with the invention, also provided is a method of protecting a substrate from the effect of ultraviolet light comprising administering an effective amount of a sunscreen composition comprising a dermatologically acceptable carrier and a cannabis extract, wherein the composition protects a substrate from the effects of ultraviolet light. In one embodiment, the cannabis extract comprises a mixture of cannabinoids. In another embodiment, the mixture of cannabinoids comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran. In still another embodiment, the mixture of cannabinoids comprises any two components selected from tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

In other embodiments, the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:20, about 1:10, or about 1:1.

In one embodiment, the mixture of cannabinoids comprises tetrahydrocannabinol and cannabidiol. In another embodiment, the mixture of cannabinoids comprises tetrahydrocannabinol and cannabidiol in a ratio of 3:1.

In other embodiments, the substrate is the skin of a subject in need thereof, and administration is a topical administration.

### DETAILED DESCRIPTION OF THE INVENTION

The following description is provided to enable any person skilled in the art to make and use the invention and sets forth the best modes contemplated by the inventors of carrying out their invention. Various modifications, however, will remain readily apparent to those skilled in the art, since the general principles of the present invention have been defined herein specifically to provide an improved sunblock or sunscreen composition.

Embodiments of the invention are discussed in detail below. In describing embodiments of the invention, specific terminology is employed for the sake of clarity. However, the invention is not intended to be limited to the specific terminology so selected. While specific exemplary embodiments are discussed, it should be understood that this is done for illustration purposes only. A person skilled in the relevant art will recognize that other components and configurations can be used without parting from the spirit and scope of the invention. All references cited herein are incorporated by reference as if each had been individually incorporated.

The term “sunscreen” or “sunblock” is used to refer to a chemical compound or chemical composition that can block or absorb UV radiation in the wavelength range of about 290 nm to about 400 nm.

### Cannabinoid Compounds

A cannabinoid is defined as a member of a class of diverse chemical compounds that interact with the endocannabinoid system in living cells. The present invention includes all naturally derived and synthetic cannabinoids, including but not limited to THC (tetrahydrocannabinol), THCA (tetrahydrocannabinolic acid), CBD (cannabidiol), CBDA (cannabidiolic acid), CBN (cannabinol), CBG (cannabigerol), CBC (cannabichromene), CBL (cannabicyclol), CBV (cannabivarin), THCV (tetrahydrocannabivarin), CBDV (cannabidivarin), CBCV (c annabichromevarin), CBGV (cannabigerovarin), CBGM (cannabigerol monomethyl ether), CBE (cannabielsoin), and CBT (cannabicitran), for example. The composition may comprise a cannabinoid or a mixture of two or more of THC, CBD, CBC, CBG, or CBDV, among other compounds. In one embodiment, the sunblock or sunscreen composition may comprise THC or CBD. In another embodiment, the sunblock or sunscreen composition may include THC and CBD.

### UV Spectrum

Ultraviolet radiation is classified into three regions: 320 to 400 nm (UVA), 290 to 320 nm (UVB) and from 200 to 290 nm (UVC). UVA is further classified into UVA I, which ranges from 340 to 400 nm, and UVA II, which ranges from 320 nm to 340 nm.

### Sun Protection Factor (SPF)

The sun protection factor (SPF) can be measured in vivo by applying sunscreen or sunblock to the skin of a subject and measuring how long it takes before sunburn occurs when exposed to an artificial sunlight source. SPF is a measure of the fraction of sunburn-producing UV rays that reach the skin. For example, “SPF 15” means that 1/15th of the burning radiation will reach the skin, assuming sunscreen is applied in an even layer at a dosage of 2 milligrams per square centimeter (mg/cm²). A user can determine the effectiveness of a sunscreen by multiplying the SPF factor by the length of time it takes for the subject to suffer a burn without sunscreen.

SPF may be measured in vitro by a spectrometer. Measurement of SPF in vitro requires measuring the transmittance of a sunscreen composition over all wavelengths in the UVB-UVA range (290-400 nm), along with a table of how effective various wavelengths are in causing sunburn (the erythemal action spectrum) and the standard intensity spectrum of sunlight.

### Boots Star Ratio System

Another approach to determine UVA protection in vitro uses a thin film technique to measure the UVA/UVB ratio. This technique addresses the problem of operator dependence by requiring only a relative measure of the UVB versus UVA absorbance of the sunscreen or sunblock product (Moyal D, Expert Rev Dermatol. 2008; 3(3):307-313). The measurement assesses the ‘flatness’ of the spectral absorbance of the product across the UVB and UVA. The absorbance of the product is summed for the UVB and UVA ranges and the UVA/UVB absorbance ratio is calculated to classify products into five classes of protection level, where the closer the ratio is towards unity, the higher the rating of the product. For example, a UVA/UVB ratio of greater than 0.91 is labeled as “Ultra” or “5.”

### Critical Wavelength

Measuring the Critical Wavelength is a method used to measure the “breadth” of UV protection. In this method, the absorbance of a thin film of the sunscreen or sunblock is integrated (summed) from 290 nm across the UV spectrum until the sum reaches 90% of the total absorbance of the sunscreen in the ultraviolet region (290-400 nm). The wavelength at which the summed absorbance reaches 90% of total absorbance is defined as the ‘critical wavelength’ and is considered to be a measure of the breadth of sunscreen or sunblock protection (Moyal D, Expert Rev Dermatol. 2008; 3(3):307-313). In one embodiment, a sunscreen or sunblock may be classified as ‘broad spectrum’ if the critical wavelength is longer than 370 nm.

A sunscreen or sunblock composition as described herein may include a sunscreen or sunblock lotion or any other composition for topical application. For example, the compositions of the invention may be anhydrous, or in an emulsion form. If in an emulsion form, the emulsions may be water-in-oil or oil-in-water. Suitable water-in-oil or oil-in-water emulsions may contain about 0.1-99%, preferably about 0.5-85%, more preferably about 5-80% by weight of oil.

In one embodiment, a sunscreen or sunblock composition may comprise one or more of a thickening agent, a viscosity modifier, an emulsifier, a colorant, a UV blocking substance selected from titanium oxide or zinc oxide, a filler, an odorant, tetrahydrocannabinol (THC), and cannabidiol (CBD). In another embodiment, a sunscreen or sunblock composition may comprise sesame oil, coconut glyceride, candilla wax, jojoba oil, shea butter, red raspberry seed oil, zinc oxide, titanium dioxide, cannabinoid compound, vanilla bean oil, and iron oxide.

In one embodiment, the sunscreen or sunblock composition may comprise between about 12-15% weight/weight (w/w) of jojoba oil. In another embodiment, the sunscreen or sunblock composition may comprise between about 6-12% or about 15-30% weight/weight (w/w) of jojoba oil

In one embodiment, the sunscreen or sunblock composition may comprise between about 25-30% weight/weight (w/w) of sesame seed oil. In another embodiment, the sunscreen or sunblock composition may comprise between about 12-25% or about 30-60% weight/weight (w/w) of sesame seed oil.

In one embodiment, the sunscreen or sunblock composition may comprise between about 3-5% weight/weight (w/w) of red raspberry oil. In another embodiment, the sunscreen or sunblock composition may comprise between about 1-3% or about 5-10% weight/weight (w/w) of red raspberry oil.

In one embodiment, the sunscreen or sunblock composition may comprise between about 5-8% weight/weight (w/w) of candelilla wax. In another embodiment, the sunscreen or sunblock composition may comprise between about 2-5% or about 8-16% weight/weight (w/w) of candelilla wax.

In one embodiment, the sunscreen or sunblock composition may comprise between about 10-15% weight/weight (w/w) of zinc oxide. In another embodiment, the sunscreen or sunblock composition may comprise between about 5-10% or about 15-30% weight/weight (w/w) of zinc oxide.

In one embodiment, the sunscreen or sunblock composition may comprise between about 10-15% weight/weight (w/w) of titanium dioxide. In another embodiment, the sunscreen or sunblock composition may comprise between about 5-10% or about 15-30% weight/weight (w/w) of titanium dioxide.

In one embodiment, the sunscreen or sunblock composition may comprise between about 12-15% weight/weight (w/w) of shea butter. In another embodiment, the sunscreen or sunblock composition may comprise between about 6-12% or about 15-30% weight/weight (w/w) of shea butter.

In one embodiment, the sunscreen or sunblock composition may comprise between about 5-8% weight/weight (w/w) of softisan. In another embodiment, the sunscreen or sunblock composition may comprise between about 2-5% or about 8-16% weight/weight (w/w) of softisan.

In one embodiment, the sunscreen or sunblock composition may comprise between about 0.2-1.0% weight/weight (w/w) of vanilla bean oil. In another embodiment, the sunscreen or sunblock composition may comprise between about 0.1-0.2% or about 1.0-2.0% weight/weight (w/w) of vanilla bean oil.

In one embodiment, the sunscreen or sunblock composition may comprise between about 1-3% weight/weight (w/w) of iron oxide (AF:Yellow). In another embodiment, the sunscreen or sunblock composition may comprise between about 0.5-1% or about 3-6% weight/weight (w/w) of iron oxide (AF:Yellow).

In one embodiment, the sunscreen or sunblock composition may comprise between about 1-3% weight/weight (w/w) of iron oxide (AF:Brown). In another embodiment, the sunscreen or sunblock composition may comprise between about 0.5-1% or about 3-6% weight/weight (w/w) of iron oxide (AF:Brown).

In one embodiment, the sunscreen or sunblock composition may comprise between about 1-3% weight/weight (w/w) of iron oxide (AF:Green). In another embodiment, the sunscreen or sunblock composition may comprise between about 0.5-1% or about 3-6% weight/weight (w/w) of iron oxide (AF:Green).

In one embodiment, the sunscreen or sunblock composition may comprise cannabinoids at 0.8% weight/weight (w/w). In another embodiment, the sunscreen or sunblock composition may comprise cannabinoids at about 0.01. 0.1, 0.5, 0.6, 0.7, 0.8, 0.9, 1, 1.1, 1.2, 1.3, 1.4, 1.5, 1.6, 2, 3, 4, 5, 6, 7, 8, 10, 15, or 20% weight/weight (w/w), or any number in between. In still another embodiment, the sunscreen or sunblock composition may comprise a mixture of two or more specific cannabinoid compounds. In still another embodiment, the sunscreen or sunblock composition may comprise THC at about 0.01. 0.1, 0.5, 0.6, 0.7, 0.8, 0.9, 1, 1.1, 1.2, 1.3, 1.4, 1.5, 1.6, 2, 3, 4, 5, 6, 7, 8, 10, 15, or 20% weight/weight (w/w), or any number in between. In yet another embodiment, the sunscreen or sunblock composition may comprise CBD at about 0.01. 0.1, 0.5, 0.6, 0.7, 0.8, 0.9, 1, 1.1, 1.2, 1.3, 1.4, 1.5, 1.6, 2, 3, 4, 5, 6, 7, 8, 10, 15, or 20% weight/weight (w/w), or any number in between.

In one embodiment, a sunscreen or sunblock composition as described herein may comprise a cannabinoid compound, or a combination of cannabinoid compounds, at a concentration of about 0.1 mg/mL to about 200 mg/mL. In another embodiment, a sunscreen or sunblock composition as described herein may comprise a cannabinoid compound, or a combination of cannabinoid compounds, at a concentration of about 0.1, 0.2, 0.3, 0.4, 0.5, 0.6, 0.7, 0.8, 0.9, 1, 2, 3, 4, 5, 6, 7, 8, 9, 10, 20, 30, 40, 50, 60, 70, 80, 90, 100, or 200 mg/mL, or any number in between.

In one embodiment, a sunscreen or sunblock composition as described herein may comprise a combination of two cannabinoid compounds at specific ratios such as 1:1, 1:2, 1:3, 1:4, 1:5, 1:6, 1:7, 1:8, 1:9, or 1:10, for example.

Sunscreen compositions according to the present invention can be formulated to achieve a variety of different SPFs. For example, the sunscreen formulations can have an in vitro or in vivo SPF of at least 5, 10, 15, 20, 25, 30, 35, 40, 45, 50, 55, 60, 65, 70, 75, 80, 85, 90, 95, 100, 105, 110, 115, 120, 125, 135, 150, 175, 200, 225, 250, 300. 325, 350, or higher, or in a range between any of these values.

Any alterations and further modifications of the compositions and/or formulations described herein, which would normally occur to one skilled in the relevant art and having possession of this disclosure, are to be considered within the scope of the instant claims.

### EXAMPLES

### Example 1

Sunscreen Formulation Overview

Divios Sunblock product is available as two main versions. Each version is differentiated by cannabinoid type and degree of purification method. Both versions contain the same target cannabinoid concentration of 8 mg/mL and include Titanium Dioxide and Zinc Oxide.

Version 1. CBD:THC (1:3, w/w) from Distillate
a. Purity is typically ˜80-90% of target cannabinoid (e.g. delta-9-THC). The remainder is other plant materials such as flavonoids, terpenoids, fatty acids etc.
b. Physical properties: highly viscous amorphous solid at room temperature. For example, the viscosity may be 100,000 centipoise (cP).

Version 2. CBD from Isolate
a. Purity is >99%
b. Physical properties: white, crystalline powder.

Physicochemical Considerations

An overview of the physicochemical property differences between THC and CBD is provided in the table below. Based on higher lipophilicity (Log P) and lower solubility of THC, THC was believed to be effective at blocking ultraviolet (UV) rays.

TABLE 1
Physicochemical differences between THC and CBD

| Characteristic | Δ9-THC | CBD |
|---|---|---|
| Molecular weight (g/mol) | 314.5 | 314.5 |
| Lipophilicity (Log P) | 6.97 | 6.10 |
| Aqueous solubility (mg/mL) | 0.003 | 0.010 |

While studies report that cannabinoids are sensitive to photo-oxidation, it was believed that UV exposure of the cannabinoid containing sunblock would not degrade at a rate that would negatively affect is UV protection properties, according to the indicated use.

For the formulation containing distillate (version 1), CBD was believed to penetrate more effectively and reside beneath THC in the epidermis. While CBD was mainly incorporated to reduce reddening and inflammation following sun exposure, it's physicochemical properties also make it suitable for UV protection. CBD has a higher potential for dermal absorption, which is considered a negative property for sunblock. The higher potential for dermal absorption of CBD was circumvented by formulation excipient and process conditions, as demonstrated in version 2.

Formulation

The sunblock target product profile (TPP) was the following:
All natural and ocean reef safe excipients.
Animal and vegan friendly.
Non-nanoparticle Titanium Dioxide and Zinc Oxide.
Minimum of SPF 25.

Cannabinoid Ratio and Concentration Selection

Based on the physicochemical considerations detailed above, the target ratios of the distillate version were CBD:THC (1:3, w/w). For cannabinoid concentration selection, a higher concentration of cannabinoids was believed to increase UV protection.

Excipient Selection

Two kinds of sunblocks, Titanium Dioxide and Zinc Oxide, were selected. Sunblocks comprising Titanium Dioxide and Zinc Oxide, because of scattering effect, often exhibit “whitening” phenomenon when applied to the skin, which seriously affects the aesthetics and the efficacy of the sunblock (Korac R and Khambholja K M. Pharmacogn Rev 5(2011); 164-173). Thus, two formulations of the sunblock were created resulting in a tinted and a non-tinted (white) formulation.

Presence of a high concentration of plant peptides on a skin surface protects peptide bonds of the skin proteins. Further, plant oils contain natural sunscreens. For example, sesame oil blocks 30% of UV rays, whereas coconut, peanut, olive, and cottonseed oils block out about 20% (Burgess C M. Chapter 70: Cosmetic products. In: Kelly A P, Taylor S C, editors. Dermatology for Skin of Color. New York: McGraw-Hill Medical; 2009). Moreover, excipients with unsaturated bonds will typically also increase UV protection. However, if the excipient is highly unsaturated, then it may cleave or undergo other photochemical reactions. These properties were considered in the selection of excipients, some of which were believed to have UV blocking properties (Viljoen J M et al. Drug Dev Ind Pharm 41(2015) 2045-2054). The resulting formulation details are provided in the table below. These weight ranges are applicable to both distillate (version 1) and isolate (version 2).

TABLE 2
Sunscreen or Sunblock composition (Tinted vs. White)

| Material Name | Weight % (Tinted) | Weight % (White) |
|---|---|---|
| Jojoba Oil | 12-15 | 12-15 |
| Sesame Seed Oil | 25-30 | 25-30 |
| Red Raspberry Oil | 3-5 | 3-5 |
| Candelilla Wax | 5-8 | 5-8 |
| Zinc Oxide | 10-15 | 10-15 |
| Titanium Dioxide | 10-15 | 10-15 |
| Shea Butter | 12-15 | 12-15 |
| Softisan | 5-8 | 5-8 |
| Vanilla Bean Oil | 0.2-1.0 | 0.2-1.0 |
| Iron Oxide AF: Yellow | 1-3 | — |
| Iron Oxide AF: Brown | 1-3 | — |
| Iron Oxide AF: Green | 1-3 | — |
| Target cannabinoids | 0.8 | 0.8 |

Modifications/improvements to existing formulation were as follows:
1. Additional UV blocking property was gained through (a) process modification; (b) excipients and their concentration modification; (c) incorporation of cannabinoids.
2. Iron oxides were incorporated to create a tinted product.
3. Longer stability/shelf life was achieved by (a) reducing crystallization rate; (b) packaging improvements.
4. Increased safety was achieved by using source materials with lower pesticide levels.
5. Improved homogeneity was achieved by (a) eliminating phase separation; (b) crystallization.

### Example 2

### Effectiveness of Cannabinoids as a Naturally Occurring UV Blocker

Two sunscreen samples were created to test for the effectiveness of cannabinoids as a naturally occurring UV blocker. All the ingredients were the same in both samples, in the same concentrations, except 400 mg of THC was added to Sample A. Sample B contained no THC. The additional materials are as follows:

TABLE 3
Composition of Sample A and Sample B

| Material Name | Sample A Weight % | Sample B Weight % |
|---|---|---|
| Jojoba Oil | 12-15 | 12-15 |
| Sesame Seed Oil | 25-30 | 25-30 |
| Red Raspberry Oil | 3-5 | 3-5 |
| Candelilla Wax | 5-8 | 5-8 |
| Zinc Oxide | 10-15 | 10-15 |
| Titanium Dioxide | 10-15 | 10-15 |
| Shea Butter | 12-15 | 12-15 |
| Softisan | 5-8 | 5-8 |
| Vanilla Bean Oil | 0.2-1.0 | 0.2-1.0 |
| Iron Oxide AF: Yellow | 1-3 | 1-3 |
| Iron Oxide AF: Brown | 1-3 | 1-3 |
| Iron Oxide AF: Green | 1-3 | 1-3 |
| Target cannabinoids | 0.8 | — |

The two samples were analyzed using an in vitro method for SPF testing. Sunscreen sample was applied at a dosage of 2 mg per square cm on 3M Transpore tape and analyzed by a Labsphere UV Transmittance Analyzer. This instrument provides the estimated in vitro SPF using a light source which simulates solar UV. The sample size was 10.

The in vitro SPF test was conducted from 290 to 400 nanometers to cover both UVB and UVAII/UVAI wavelengths. SPF was calculated using software which integrates the area under the absorbance curve in this wavelength. The results demonstrated a significant increase in the SPF for Sample A (containing THC) over Sample B (Control).

TABLE 4
The following data was obtained from UV testing:

| Sample | Sample A | Sample B | |
|---|---|---|---|
| In vitro SPF (average) | 29.25 | 21.58 | |
| UVA/UVB ratio (average) | 0.92 | 0.9 | |
| Critical Wavelength | 386 | 386 | |
| # of data points | 10 | 10 | |
| Standard Deviation | 2.09 | 0.91 | |
| Boots Star rating | 5 (Ultra) | 5 (Ultra) | |
| Experimental Data (THC infused) | | | |
| wavelength range | 290-400 nm | | |
| Units | SPF | T(UVA) | T(UVB) |
| # of scans | 10 | 10 | 10 |
| Mean | 29.25 | 4.82% | 3.38% |
| STD (standard deviation | 2.09 | 0.33% | 0.26% |
| cov | 7.16% | 6.75% | 7.59% |
| UVA/UVB ratio | 0.92 | | |
| Boots Star Rating | ***** (Ultra) | | |
| | | | Critical |
| | Scan | SPF | Wavelength |
| | 1 | 25.21 | 385 |
| | 2 | 29.01 | 385 |
| | 3 | 30.48 | 385 |
| | 4 | 32.46 | 385 |
| | 5 | 31.87 | 386 |
| | 6 | 29.84 | 386 |
| | 7 | 28.35 | 386 |
| | 8 | 28.97 | 386 |
| | 9 | 27.55 | 386 |
| | 10 | 28.77 | 386 |
| Control Sample Data | | | |
| wavelength range | 290-400 nm | | |
| Units | SPF | T(UVA) | T(UVB) |
| # of scans | 10 | 10 | 10 |
| Mean | 21.58 | 6.59% | 4.51% |
| STD (standard deviation | 0.91 | 0.20% | 0.19% |
| cov | 4.22% | 3.04% | 4.28% |
| UVA/UVB ratio | 0.9 | | |
| Boots Star Rating | ***** (Ultra) | | |
| | 1 | 22.08 | 386 |
| | 2 | 22.63 | 386 |
| | 3 | 22.26 | 386 |
| | 4 | 20.52 | 386 |
| | 5 | 21.33 | 386 |
| | 6 | 20.88 | 386 |
| | 7 | 23.02 | 386 |
| | 8 | 21.63 | 386 |
| | 9 | 20.23 | 386 |
| | 10 | 21.27 | 386 |

The above results show that Sample A is surprisingly superior to Sample B. For example, the increase in SPF in Sample A, which includes cannabinoids, is about 50% compared to Sample B.

In some embodiments, the cannabinoids may have a synergistic effect with known UV blocking substances such as zinc oxide and titanium dioxide. For example, the combination of cannabinoids with one or more of these substances may be greater than the effects of any of those substances by itself, especially with respect to the entire UV spectrum. Some materials are only effective for a limited portion of UV wavelengths, and cannabinoids appear to be highly absorptive in parts of the UV spectrum where titanium dioxide and zinc oxide are less effective. Accordingly, a benefit of sunblock that includes cannabinoids may be a broad spectrum coverage that is not otherwise possible with conventional sunscreen or sunblock compositions.

Another benefit of sunblock that includes a cannabinoid compound such as THC and/or CBD is that these compounds are naturally occurring ingredients with reduced chance that harmful substances may be present in the additive. In addition, THC and CBD are non-toxic, reducing health risks associated with topical application and ingestion.

The following claims are thus to be understood to include what is specifically illustrated and described above, what is conceptually equivalent, what can be obviously substituted and also what essentially incorporates the essential idea of the invention. Those skilled in the art will appreciate that various adaptations and modifications of the just-described preferred embodiment can be configured without departing from the scope of the invention. The illustrated embodiment has been set forth only for the purposes of example and that should not be taken as limiting the invention. Therefore, it is to be understood that, within the scope of the appended claims, the invention may be practiced other than as specifically described herein.

What is claimed is:

1. A sunscreen composition comprising a dermatologically acceptable carrier and a cannabis extract, wherein the composition protects a substrate from the effects of ultraviolet light.

2. The sunscreen composition of claim 1, wherein the cannabis extract comprises a mixture of cannabinoids.

3. The sunscreen composition of claim 2, wherein the mixture of cannabinoids comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

4. The sunscreen composition of claim 2, wherein the mixture of cannabinoids comprises any two components selected from tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

5. The sunscreen composition of claim 4, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:20.

6. The composition of claim 4, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:10.

7. The composition of claim 4, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:1.

8. The composition of claim 4, wherein the mixture of cannabinoids comprises tetrahydrocannabinol and cannabidiol.

9. The composition of claim 8, wherein the mixture of cannabinoids comprises tetrahydrocannabinol and cannabidiol in a ratio of 3:1.

10. The sunscreen composition of claim 1, wherein the cannabis extract comprises between about 0.1 to about 20% weight/weight of the sunscreen composition.

11. The sunscreen composition of claim 1, wherein the sunscreen composition provides an SPF value of at least 5.

12. The sunscreen composition of claim 1, wherein the composition protects the substrate from the effect of ultraviolet light.

13. The sunscreen composition of claim 1, wherein the composition protects the substrate from the effect of ultraviolet A light.

14. The sunscreen composition of claim 1, wherein the composition protects the substrate from the effect of ultraviolet B light.

15. A method of protecting a substrate from the effect of ultraviolet light comprising administering an effective amount of a sunscreen composition of claim 1.

16. The method of claim 15, wherein the cannabis extract comprises a mixture of cannabinoids.

17. The method of claim 16, wherein the mixture of cannabinoids comprises tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

18. The method of claim 16, wherein the mixture of cannabinoids comprises any two components selected from tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, or cannabicitran.

19. The method of claim 18, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:20.

20. The method of claim 18, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:10.

21. The method of claim 18, wherein the mixture of cannabinoids comprises any two components selected from the group consisting of tetrahydrocannabinol, tetrahydrocannabinolic acid, cannabidiol, cannabidiolic acid, cannabinol, cannabigerol, cannabichromene, cannabicyclol, cannabivarin, tetrahydrocannabivarin, cannabidivarin, cannabichromevarin, cannabigerovarin, cannabigerol monomethyl ether, cannabielsoin, and cannabicitran, wherein the two components are in a ratio of about 1:1.

22. The method of claim 18, wherein the mixture of cannabinoids comprises tetrahydrocannabinol and cannabidiol.

23. The method of claim 22, wherein the mixture of cannabinoids comprises tetrahydrocannabinol and cannabidiol in a ratio of 3:1.

24. The method of claim 15, wherein the substrate is the skin of a subject in need thereof, and administration is a topical administration.

25. The method of claim 15, wherein the sunscreen composition provides an SPF value of at least 5.
